(19) United States
(12) Patent Application Publication
(10) Pub. No.: US 2007/0148104 A1
(43) Pub. Date: Jun. 28, 2007

(54) Animal chew articles

(76) Inventors: Edward J. Goettert, Dellwood, MN (US); Steven S. Kantner, St. Paul, MN (US)

Correspondence Address: 3M INNOVATIVE PROPERTIES COMPANY, PO BOX 33427, ST. PAUL, MN, 55133-3427, US

(21) Appl. No.: 11/317,376

(22) Filed: Dec. 23, 2005

(51) Int. Cl. A61K 8/36

(52) U.S. Cl. 424/49; 424/442

(57) ABSTRACT

Edible chew articles for animals that comprise: (a) edible chew base, (b) an effective amount of antimicrobial lipid, and (c) at least one of (1) an effective amount of tartar control agent or (2) an effective amount of a long chain fatty acid. Also a method comprising chewing or eating by an animal of such an article.

### FIELD

The invention relates to chew articles that improve dental health of animals such as pets. The invention also relates to a process for preventing tartar formation on and tartar removal from animal's teeth by chewing on such chew articles.

### BACKGROUND OF INVENTION

Dental calculus, or tartar as it is sometimes called, is a deposit which forms on the surfaces of the teeth at the gingival margin. Supragingival calculus appears principally in the areas near the orifices of the salivary ducts; e.g., on the lingual surfaces of the lower anterior teeth and on the buccal surfaces of the upper first and second molars, and on the distal surfaces of the posterior molars. Calculus develops from a biofilm precursor through calcification. This biofilm is known as plaque. Mature calculus consists of an inorganic portion which is largely calcium phosphate arranged in a hydroxylapatite crystal lattice structure similar to bone, enamel and dentine. An organic portion is also present and consists of desquamated epithelial cells, leukocytes, salivary sediment, food debris and various types of microorganisms. As the mature calculus develops, it becomes visibly white or yellowish in color unless stained or discolored by some extraneous agency. In addition to being unsightly and undesirable from an aesthetic standpoint, the mature calculus deposits are constant sources of irritation of the gingiva and thereby are a contributing factor to gingivitis and other diseases of the supporting structures of the teeth, the irritation decreasing the resistance of tissues to endogeneous and exogenous organisms.

The microorganisms that the plaque and calculus harbor not only can cause infections in the oral cavity, but also cause breath malodor due to metabolism of sulfur containing proteinaceous materials from the desquamated epithelial cells, food debris, etc. Breath malodor is a common complaint of owners of companion animals and a variety of methods have been developed to absorb or mask this odor.

A wide variety of chemical and biological agents have also been suggested in the art to retard calculus formation or to remove calculus after it is formed in humans and in animals, particularly pets. Mechanical removal of this material is done routinely in humans but is more problematic with regard to animals.

Illustrative chewing articles for pets are disclosed in, for example, German Patent No. 3,426,203 (Hans), U.S. Pat. No. 3,882,257 (Cagle), 4,145,447 (Fisher et al.), U.S. Pat. No. 5,000,943 (Scaglione et al.), U.S. Pat. No. 5,296,209 (Simone et al.), and U.S. Pat. No. 5,618,518 (Stookey).

U.S. Pat. No. 3,701,830 (Weinrich et al.), U.S. Pat. No. 4,364,925 (Fisher), U.S. Pat. No. 3,194,738 (Harrison et al.), and U.S. Pat. No. 3,686,393 (Woodruff et al.) disclose the use of enzymes for inhibiting plaque.

### SUMMARY

The present invention provides chew articles that promote dental health of the consuming animal and a process for inhibiting tartar formation on and facilitating tartar removal from the animal's teeth by chewing on such chew articles. The invention provides an easy, effective way for pet owners to maintain the oral health of their pet animals, e.g., typically cats and dogs.

The present invention employs a combination of ingredients that promote dental health of the using animal delivered in the form of a chewable article. The present invention employs antimicrobial (including, e.g., antiviral, antibacterial, and antifungal) compositions. These compositions include one or more antimicrobial lipids, such as, for example, a fatty acid ester of a polyhydric alcohol, a fatty ether of a polyhydric alcohol, or alkoxylated derivatives thereof (of either the ester or ether). In certain embodiments the compositions also include one or more tartar control components that can also serve as enhancers for the antimicrobial action of the antimicrobial lipid. In other embodiments the compositions also include one or more long chain fatty acids that can broaden the spectrum and enhance the speed of activity of the antimicrobial lipid. Compositions used in articles and processes of the invention can provide effective reduction, prevention, or elimination of microbes, particularly bacteria, fungi, and viruses. Preferably, the microbes are of a relatively wide variety such that the compositions of the present invention have a broad spectrum of activity.

In brief summary, articles of the invention comprise: (a) edible chew base, (b) an effective amount of antimicrobial lipid, and (c) at least one of, and preferably both (1) an effective amount of tartar control agent or (2) an effective amount of a long chain fatty acid. In brief summary, the process of the invention comprises chewing or eating by an animal of the article of claim 1.

In accordance with the present invention, an animal such as a pet will undergo a decolonizing at least a portion of the oral cavity of a subject of microorganisms. As a result, the dental health of the animal will be improved.

Definitions

The following terms are used herein according to the following definitions.

“Affliction” means a condition to a body resulting from sickness, disease, injury, bacterial colonization, etc.

“Antimicrobial lipid” means an antiseptic that preferably has a solubility in water of no greater than 1.0 gram per 100 grams (1.0 g/100 g) deionized water. Preferred antimicrobial lipids have a solubility in water of no greater than 0.5 g/100 g deionized water, more preferably, no greater than 0.25 g/100 g deionized water, and even more preferably, no greater than 0.10 g/100 g deionized water. Solubilities are determined using radiolabeled compounds as described under Conventional Solubility Estimations in “Solubility of Long-Chain Fatty Acids in Phosphate Buffer at pH 7.4”, Henrik Vorum et al., in Biochimica et Biophysica Acta, 1126, 135-142 (1992). Preferred antimicrobial lipids have a solubility in deionized water of at least 100 micrograms (μg) per 100 grams deionized water, more preferably, at least 500 μg/100 g deionized water, and even more preferably, at least 1000 μg/100 g deionized water. The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

“Antiseptic” means a chemical agent that kills pathogenic and non-pathogenic microorganisms. Preferred antiseptics exhibit at least a 4 log reduction of both P. aeruginosa and S. aureus in 60 minutes from an initial inoculum of 1 to 3×10⁷ cfu/ml when tested in Mueller Hinton broth at 35° C. at a concentration of 0.25 wt-% in a Rate of Kill assay using an appropriate neutralizer as described in “The Antimicrobial Activity in vitro of chlorhexidine, a mixture of isothiazolinones (Kathon CG) and cetyl trimethyl ammonium bromide (CTAB),” G. Nicoletti et al., Journal of Hospital Infection, 23, 87-111 (1993). Antiseptics generally interfere more broadly with the cellular metabolism and/or the cell envelope.

“Decolonization” refers to a reduction in the number of microorganisms (e.g., bacteria and fungi) present in or on tissue that do not necessarily cause immediate clinical symptoms. Examples of decolonization include, but are not limited to, decolonization of the oral cavity. Ordinarily fewer microorganisms are present in colonized tissue than in infected tissue. When the tissue is completely deconolonized the microorganisms have been “eradicated.”

“Effective amount” means the amount of the antimicrobial lipid component and/or the enhancer component when in a composition, as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level. It should be understood that in the compositions of the present invention, the concentrations or amounts of the components, when considered separately, may not kill to an acceptable level, or may not kill as broad a spectrum of undesired microorganisms, or may not kill as fast; however, when used together such components provide an enhanced (preferably synergistic) antimicrobial activity (as compared to the same components used alone under the same conditions).

“Enhancer” means a component that enhances the effectiveness of the antimicrobial lipid component such that when the composition less the antimicrobial lipid component and the composition less the enhancer component are used separately, they do not provide the same level of antimicrobial activity as the composition as a whole. For example, an enhancer component in the absence of the antimicrobial lipid component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antimicrobial lipid component.

“Fatty” as used herein refers to a straight or branched chain alkyl or alkylene moiety having 7 to 22 (odd or even number) carbon atoms, unless otherwise specified.

“Microorganism” or “microbe” refers to bacteria, yeast, mold, fungi, protozoa, mycoplasma, as well as viruses (including lipid enveloped RNA and DNA viruses). p “Mucous membranes,” “mucosal membranes,” and “mucosal tissue” are used interchangeably and refer to the surfaces of the nasal (including anterior nares, nasoparangyl cavity, etc.), oral (e.g., mouth), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

“Stable” means physically stable or chemically stable.

“Treat” or “treatment” means to improve the condition of a subject relative to the affliction, typically in terms of clinical symptoms of the condition.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an affliction means preventing, treating, or both treating and preventing further afflictions).

Also herein, the recitations of numerical ranges by endpoints include all numbers subsumed within that range (e.g., 1 to 5 includes 1, 1.5, 2, 2.75, 3, 3.80, 4, 5, etc.).

### DETAILED DESCRIPTION OF ILLUSTRATIVE EMBODIMENTS

As described above, pet chew articles of the invention comprise: (a) edible chew base, (b) an effective amount of antimicrobial lipid, and (c) at least one of, and preferably both (1) an effective amount of tartar control agent or (2) an effective amount of a long chain fatty acid.

Chew Base

The chew base may be made of any edible or chewable material. Illustrative examples include rawhides, biscuits of a variety of compositions, etc.

The chew base should be safe for pets to chew and swallow. Preferably it is of appropriate strength, texture, and firmness to be desirable as a pet chew toy.

The characteristics of the chew base typically define much of the physical properties of the chew article, including substantially defining the density, frangibility, stiffness, and strength of the chew article. Accordingly, it is typically desired to select a chew base that is suitable for forming desired shapes for the chew article. Some illustrative shapes of chew articles of the invention include bones, rings, substantially cylindrical sticks, wafers, biscuits, etc.

A preferred chew base is so-called modified wheat gluten. Articles made with such chew base can be made with desirable hardness and do not get “gummy” or “slimy” when wet. In addition, such chew base can be readily molded into desired shape. A second suitable chew base is gelatinized starch. Illustrative examples of suitable chew base compositions are disclosed in U.S. Pat. Nos. 5,747,648, 5,965,708, and 5,977,312 (all Bassi et al.), and U.S. Patent Application Publications 2004/0086616, 2004/0197455, 2005/0008759, and 2005/0214349 (all Nie et al.). As disclosed in those documents, starch and gluten products can be made with very desirable strength. Moreover, such products can be readily controlled in terms of color and opacity.

The chew base is preferably a tough, pliant material that will provide the animal using it with vigorous mechanical action against the teeth as well as entertainment value.

The other components of the article are blended into the chew base, e.g., by mixing, typically before molding or otherwise shaping the article.

Antimicrobial Lipid

Chew articles of the invention comprise an antimicrobial lipid component. In certain embodiments, the antimicrobial lipid component includes a fatty acid ester of a polyhydric alcohol, a fatty ether of a polyhydric alcohol, alkoxylated derivatives thereof (of either the ester or ether), or combinations thereof.

The antimicrobial lipid component is that component of the composition that provides at least part of the antimicrobial activity. That is, the antimicrobial lipid component has at least some antimicrobial activity for at least one microorganism. It is generally considered the main active component of the compositions of the present invention.

In one embodiment, the lipid component includes a C₇ to C₁₄ saturated fatty acid ester of a polyhydric alcohol, a C₈ to C₂₂ unsaturated fatty acid ester of a polyhydric alcohol, a C₇ to C₁₄ saturated fatty ether of a polyhydric alcohol, a C₈ to C₂₂ unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters include monoesters and the ethers include monoethers, and for sucrose the esters include monoesters, diesters, or combinations thereof, and the ethers include monoethers, diethers, or combinations thereof.

Preferably, the antimicrobial lipid component is present in an amount of at least 0.1 wt-%. Unless otherwise specified, all weight percents are based on the total weight of a “ready to use” or “as used” composition. Preferably, if the antimicrobial lipid component includes a monoester of a polyhydric alcohol, a monoether of a polyhydric alcohol, or an alkoxylated derivative thereof, then there is no more than 50 wt-%, more preferably no more than 40 wt-%, even more preferably no more than 25 wt-%, and even more preferably no more than 15 wt-% of a diester, diether, triester, triether, or alkoxylated derivative thereof present, based on the total weight of the antimicrobial lipid component.

Preferably, the antimicrobial lipid component includes glycerol monolaurate, glycerol monocaprate, glycerol monocaprylate, propylene glycol monolaurate, propylene glycol monocaprate, propylene glycol monocaprylate, and combinations thereof.

Perferably the antimicrobial lipid component is recognized as a food additive by the United States Food and Drug Administration and is used in an amount not exceeding the amount permitted by the FDA.

The antimicrobial lipids preferably have a hydrophile/lipophile balance (HLB) of at most 6.2, more preferably at most 5.8, and even more preferably at most 5.5. The antimicrobial lipids preferably have an HLB of at least 3, preferably at least 3.2, and even more preferably at least 3.4.

Preferred antimicrobial lipids are uncharged and have an alkyl or alkenyl hydrocarbon chain containing at least 7 carbon atoms.

In one embodiment, the present invention provides a method of decolonizing at least a portion of the oral cavity of a subject of microorganisms. The method includes contacting the oral cavity with an antimicrobial composition in an amount effective to kill one or more microorganisms, wherein the antimicrobial composition includes: an effective amount of an antimicrobial lipid component that includes a C₇ to C₁₄ saturated fatty acid ester of a polyhydric alcohol, a C₈ to C₂₂ unsaturated fatty acid ester of a polyhydric alcohol, a C₇ to C₁₄ saturated fatty ether of a polyhydric alcohol, a C₈ to C₂₂ unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters include monoesters and the ethers include monoethers, and for sucrose the esters include monoesters, diesters, or combinations thereof, and the ethers include monoethers, diethers, or combinations thereof, and optionally, an effective amount of an enhancer component that includes an alpha-hydroxy acid, a beta-hydroxy acid, a chelating agent, a C₁ to C₄ alkyl carboxylic acid, a C₆ to C₁₂ aryl carboxylic acid, a C₆ to C₁₂ aralkyl carboxylic acid, a C₆ to C₁₂ alkaryl carboxylic acid, a phenolic compound, a C₁ to C₁₀ alkyl alcohol, an ether glycol, or combinations thereof.

In one embodiment, the present invention provides a method of decolonizing at least a portion of the oral cavity of a subject of microorganisms, the method including contacting the oral cavity with an antimicrobial composition in an amount effective to kill one or more microorganisms, wherein the antimicrobial composition includes: an effective amount of an antimicrobial lipid component having a solubility in water of at least 100 μg/100 g deionized water and at most 1 g/100 g deionized water.

In other embodiments, the present invention provides methods for killing or inactivating microorganisms. Herein, to “kill or inactivate” means to render the microorganism ineffective by killing them (e.g., bacteria and fungi) or otherwise rendering them inactive (e.g., viruses). The present invention provides methods for killing bacteria such as Staphylococcus spp., Streptococcus spp., Escherichia spp., Enterococcus spp., Pseudamonas spp. bacteria and combinations thereof, and more particularly Streptococcus mutans, Actinomyces viscosus, and Fusobacterium nucleatum, and combinations thereof which often are in the oral cavity of a subject. The method includes contacting the microorganism with an antimicrobial composition of the present invention in an amount effective to kill one or more microorganisms (e.g., bacteria and fungi) or inactivate one or more microorganisms (e.g., viruses).

For certain applications in which limited antimicrobial activity is desired, compositions containing an antimicrobial lipid component can be used, whereas in other applications in which more broad antimicrobial activity is desired, compositions containing both an antimicrobial lipid component and an enhancer component are used. For example, in certain situations it may be desirable to kill or inactivate only one type or class of microorganism (e.g., Gram positive) as opposed to all the microorganisms present. In such situations, compositions of the present invention that contain an antimicrobial lipid component without an enhancer component may be suitable.

Those of ordinary skill in the art will readily determine when a composition of the present invention provides antimicrobial activity using assay and bacterial screening methods well known in the art. One readily performed assay involves exposing selected known or readily available viable microorganism strains, such as Enterococcus spp., Aspergillus spp., Escherichia spp., Staphylococcus spp., Streptococcus spp., Pseudomonas spp., or Salmonella spp., to a test composition at a predetermined bacterial burden level in a culture media at an appropriate temperature. Briefly, after a sufficient contact time, an aliquot of a sample containing the exposed bacteria is collected, diluted, and plated out on agar. The plated sample of bacteria is incubated for forty-eight hours and the number of viable bacterial colonies growing on the plate is counted. Once colonies have been counted the reduction in the number of bacteria caused by the test composition is readily determined. Bacterial reduction is generally reported as log₁₀ reduction determined by the difference between the log₁₀ of the initial inoculum count and the log₁₀ of the inoculum count after exposure. Preferred compositions of the present invention have an average of at least a 4 log reduction in test bacteria in 10 minutes.

Significantly, certain embodiments of the present invention have a very low potential for generating microbial resistance. For example, preferred compositions of the present invention have an increase in the ratio of final to initial MIC levels (i.e., minimum inhibitory concentration) of less than 16, more preferably less than 8, and even more preferably less than 4. Such an emergence of resistance assay should be carried out such that the microorganisms are subjected initially to sub MIC levels (e.g., ½ the MIC) of antimicrobial lipid and after 24 hours the microorganisms passed into broth containing twice the concentration of antimicrobial lipid. This is repeated for 8 days and each day microorganisms are removed to determine the new MIC. Thus, such compositions can be provided to the animal several times over one or more days to improve dental health or to eradicate unwanted bacteria (such as oral colonization of Streptococcus mutans).

Preferred compositions of the present invention contain an effective amount of antimicrobial lipid component to rapidly kill or inactivate microorganisms on teeth and the mucosal membranes of the oral cavity. In certain embodiments, essentially all the microorganisms are eradicated or inactivated within five days, preferably within three days, more preferably two days, and most preferably within 24 hours using one or more chewable articles per day.

Preferred compositions of the present invention have a generally low irritation level for skin, skin lesions, and mucosal membranes (including the oral cavity).

In certain embodiments, the antimicrobial lipid component preferably includes one or more fatty acid esters of a polyhydric alcohol, fatty ethers of a polyhydric alcohol, or alkoxylated derivatives thereof (of either or both of the ester and ether), or combinations thereof. More specifically and preferably, the antimicrobial component is selected from the group consisting of a C₇ to C₁₄ saturated fatty acid ester of a polyhydric alcohol (preferably, a C₈ to C₁₂ saturated fatty acid ester of a polyhydric alcohol), a C₈ to C₂₂ unsaturated fatty acid ester of a polyhydric alcohol (preferably, a C₁₀ to C₂₂ unsaturated fatty acid ester of a polyhydric alcohol), a C₇ to C₁₄ saturated fatty ether of a polyhydric alcohol (preferably, a C₈ to C₁₂ saturated fatty ether of a polyhydric alcohol), a C₈ to C₂₂ unsaturated fatty ether of a polyhydric alcohol (preferably, a C₁₀ to C₂₂ unsaturated fatty ether of a polyhydric alcohol), an alkoxylated derivative thereof, and combinations thereof. Preferably, the esters and ethers are monoesters and monoethers, unless they are esters and ethers of sucrose in which case they can be monoesters, diesters, monoethers, or diethers. Various combinations of monoesters, diesters, monoethers, and diethers can be used in a composition of the present invention.

A fatty acid ester of a polyhydric alcohol is preferably of the formula (R¹—C(O)—O)ₙ—R², wherein R¹ is the residue of a C₇ to C₁₄ saturated fatty acid (preferably, a C₈ to C₁₂ saturated fatty acid), or a C₈ to C₂₂ unsaturated (preferably, a C₁₀ to C₂₂ unsaturated, including polyunsaturated) fatty acid, R² is the residue of a polyhydric alcohol (typically and preferably, glycerin, propylene glycol, and sucrose, although a wide variety of others can be used including pentaerythritol, sorbitol, mannitol, xylitol, etc.), and n is 1 or 2. The R² group includes at least one free hydroxyl group (preferably, residues of glycerin, propylene glycol, or sucrose). Preferred fatty acid esters of polyhydric alcohols are esters derived from C₇, C₈, C₉, C₁₀, and C₁₂ saturated fatty acids. For embodiments in which the polyhydric alcohol is glycerin or propylene glycol, n is 1, although when it is sucrose, n is 1 or 2.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachidonic (20:4) unsaturated (including polyunsaturated) fatty acids. As is generally know, 18:1, for example, means the compound has 18 carbon atoms and 1 carbon-carbon double bond. Preferred unsaturated chains have at least one unsaturated group in the cis isomer form. In certain preferred embodiments, the fatty acid monoesters that are suitable for use in the present composition include known monoesters of lauric, caprylic, and capric acid, such as that known as GML or the trade designation LAURICIDIN™ (the glycerol monoester of lauric acid commonly referred to as monolaurin or glycerol monolaurate), glycerol monocaprate, glycerol monocaprylate, propylene glycol monolaurate, propylene glycol monocaprate, propylene glycol monocaprylate, and combinations thereof.

Exemplary fatty acid diesters of sucrose include, but are not limited to, lauric, caprylic, and capric diesters of sucrose as well as combinations thereof.

A fatty ether of a polyhydric alcohol is preferably of the formula (R³O)ₙR⁴, wherein R³ is a C₇ to C₁₄ saturated aliphatic group (preferably, a C₈ to C₁₂ saturated aliphatic group), or a C₈ to C₂₂ unsaturated (preferably, C₁₀ to C₂₂ unsaturated, including polyunsaturated) aliphatic group, R⁴ is the residue of glycerin, sucrose, or propylene glycol, and n is 1 or 2. For glycerin and propylene glycol n is 1, and for sucrose n is 1 or 2. Preferred fatty ethers are monoethers of C₇ to C₁₄ alkyl groups (more preferably, C₈ to C₁₂ alkyl groups).

Exemplary fatty monoethers include, but are not limited to, laurylglyceryl ether, caprylglyceryl ether, caprylylglyceryl ether, laurylpropylene glycol ether, caprylpropylene glycol ether, and caprylylpropylene glycol ether. Other fatty monoethers include glycerin and propylene glycol monoethers of oleyl (18:1), linoleyl (18:2), linolenyl (18:3), and arachidonyl (20:4) unsaturated and polyunsaturated fatty alcohols. In certain preferred embodiments, the fatty monoethers that are suitable for use in the present composition include laurylglyceryl ether, caprylglyceryl ether, caprylylglyceryl ether, laurylpropylene glycol ether, caprylpropylene glycol ether, caprylylpropylene glycol ether, and combinations thereof. Unsaturated chains preferably have at least one unsaturated bond in the cis isomer form.

The alkoxylated derivatives of the aforementioned fatty acid esters and fatty ethers (e.g., one which is ethoxylated and/or propoxylated on the remaining alcohol group(s)) also have antimicrobial activity as long as the total alkoxylate is kept relatively low. Preferred alkoxylation levels are disclosed in U.S. Pat. No. 5,208,257 (Kabara). In the case where the esters and ethers are ethoxylated, the total moles of ethylene oxide is preferably less than 5, and more preferably less than 2.

The fatty acid esters or fatty ethers of polyhydric alcohols can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The compositions of the present invention include one or more fatty acid esters, fatty ethers, alkoxylated fatty acid esters, or alkoxylated fatty ethers at a suitable level to produce the desired result. Such compositions preferably include a total amount of such material of at least 0.01 percent by weight (wt-%), more preferably at least 0.1 wt-%, even more preferably at least 0.25 wt-%, even more preferably at least 0.5 wt-%, and even more preferably at least 1 wt-%, based on the total weight of the “ready to use” or “as used” composition. In a preferred embodiment, they are present in a total amount of no greater than 20 wt-%, more preferably no greater than 15 wt-%, even more preferably no greater than 10 wt-%, and even more preferably no greater than 5 wt-%, based on the “ready to use” or “as used” composition.

Preferred compositions of the present invention that include one or more fatty acid monoesters, fatty monoethers, or alkoxylated derivatives thereof can also include a small amount of a di-or tri-fatty acid ester (i.e., a fatty acid di- or tri-ester), a di-or tri-fatty ether (i.e., a fatty di-or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antimicrobial lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antimicrobial lipid components present in the composition.

Although in some situations it is desirable to avoid di-or tri-esters as a component of the starting materials, it is possible to use relatively pure tri-esters in the preparation of certain compositions of the present invention (for example, as a nutritional and flavor enhancing fatty component) and have effective antimicrobial activity.

Typically chew articles of the invention comprise at least one of (1) an effective amount of tartar control agent or (2) an effective amount of long chain fatty acid as an enhancer. Preferably, a chew article will include both components.

Tartar Control Agent

Illustrative examples of tartar control agents that can be used herein include an alpha-hydroxy acid, a beta-hydroxy acid, and a chelating agent in a matrix of edible proteinaceous material that provides long lived chewing and mechanic cleaning of tooth surfaces.

In certain embodiments, the tartar control component preferably includes a carboxylic acid. In certain embodiments, the tartar control component preferably includes an alpha-hydroxy acid. In certain embodiments, the tartar control component preferably includes citric acid. In certain embodiments, the tartar control component preferably includes a chelator. In certain embodiments, the tartar control component preferably includes hexametaphosphoric acid and its salts and pyrophosphoric acid and its salts. The tartar control component also serves to enhance the antimicrobial activity especially against Gram negative bacteria, such as E. coli and Pseudomonas sp.

One or more tartar control components may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition.

Alpha-hydroxy Acids. An alpha-hydroxy acid is typically a compound represented by the formula:
R⁵(CR⁶OH)ₙCOOH
wherein: R⁵ and R⁶ are each independently H or a C₁ to C₈ alkyl group (straight, branched, or cyclic), a C₆ to C₁₂ aryl, or a C₆ to C₁₂ aralkyl or alkaryl group (wherein the alkyl group is straight, branched, or cyclic), wherein R⁵ and R⁶ may be optionally substituted with one or more carboxylic acid groups; and n is 1 to 3, preferably, 1 to 2.

Exemplary alpha-hydroxy acids include, but are not limited to, lactic acid, malic acid, citric acid, 2-hydroxybutanoic acid, mandelic acid, gluconic acid, glycolic acid, tartaric acid, ascorbic acid, and alpha-hydroxyoctanoic acid, as well as derivatives thereof (e.g., compounds substituted with hydroxyls, phenyl groups, hydroxyphenyl groups, alkyl groups, halogens, as well as combinations thereof). Preferred alpha-hydroxy acids include lactic acid, malic acid, mandelic acid, and citric acid. These acids may be in D, L, or DL form and may be present as free acid, lactone, or partial salts thereof. All such forms are encompassed by the term “acid.” Preferably, the acids are present in the free acid form. In certain preferred embodiments, the alpha-hydroxy acids useful in the compositions of the present invention are selected from the group consisting of lactic acid, mandelic acid, malic acid, citric acid, and mixtures thereof. Other suitable alpha-hydroxy acids are described in U.S. Pat. No. 5,665,776 (Yu).

One or more alpha-hydroxy acids may be used in the compositions of the present invention at a suitable level to produce the desired result. In a preferred embodiment, they are present in a total amount of at least 0.25 wt-%, more preferably, at least 0.5 wt-%, and even more preferably, at least 1 wt-%, based on the total weight of the ready to use composition. In a preferred embodiment, they are present in a total amount of no greater than 10 wt-%, more preferably, no greater than 5 wt-%, and even more preferably, no greater than 3 wt-%, based on the total weight of the ready to use composition. Higher concentrations may become irritating.

Beta-hydroxy Acids. A beta-hydroxy acid is typically a compound represented by the formula: 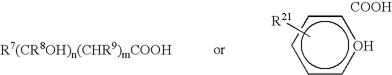
wherein: R⁷, R⁸, and R⁹ are each independently H or a C₁ to C₈ alkyl group (saturated straight, branched, or cyclic group), a C₆ to C₁₂ aryl, or a C₆ to C,₂ aralkyl or alkaryl group (wherein the alkyl group is straight, branched, or cyclic), wherein R₇ and R₈ may be optionally substituted with one or more carboxylic acid groups; m is 0 or 1; n is 1 to 3, preferably, 1 or 2; and R²¹ is H, a C1 to C4 alkyl, or a halogen.

Exemplary beta-hydroxy acids include, but are not limited to, salicylic acid, beta-hydroxybutanoic acid, tropic acid, and trethocanic acid. In certain preferred embodiments, the beta-hydroxy acids useful in the compositions of the present invention are selected from the group consisting of salicylic acid, beta-hydroxybutanoic acid, and mixtures thereof. Other suitable beta-hydroxy acids are described in U.S. Pat. No. 5,665,776 (Yu).

One or more beta-hydroxy acids may be used in the compositions of the present invention at a suitable level to produce the desired result. In a preferred embodiment, they are present in a total amount of at least 0.1 wt-%, more preferably at least 0.25 wt-%, and even more preferably at least 0.5 wt-%, based on the total weight of the ready to use composition. In a preferred embodiment, they are present in a total amount of no greater than 10 wt-%, more preferably no greater than 5 wt-%, and even more preferably no greater than 3 wt-%, based on the total weight of the ready to use composition. Higher concentrations may become irritating.

Chelators. A chelating agent (i.e., chelator) is typically an organic compound capable of multiple coordination sites with a metal ion in solution. Typically these chelating agents are polyanionic compounds and coordinate best with polyvalent metal ions. Exemplary chelating agents include, but are not limited to, ethylene diamine tetraacetic acid (EDTA) and salts thereof (e.g., EDTA(Na)₂, EDTA(Na)₄, EDTA(Ca), EDTA(K)₂), sodium acid pyrophosphate, adipic acid, succinic acid, polyphosphoric acid, sodium hexametaphosphate, acidified sodium hexametaphosphate, nitrilotris(methylenephosphonic acid), diethylenetriaminepentaacetic acid, 1-hydroxyethylene-1,1 -diphosphonic acid, and diethylenetriaminepenta-(methylenephosphonic acid). Certain carboxylic acids, particularly the alpha-hydroxy acids and beta-hydroxy acids, can also function as chelators, e.g., malic acid and tartaric acid.

One or more chelating agents may be used in the compositions of the present invention at a suitable level to produce the desired result. In a preferred embodiment, they are present in a total amount of at least 0.01 wt-%, more preferably at least 0.05 wt-%, even more preferably at least 0.1 wt-%, and even more preferably at least 1 wt-%, based on the weight of the ready to use composition. In a preferred embodiment, they are present in a total amount of no greater than 10 wt-%, more preferably no greater than 5 wt-%, and even more preferably no greater than 1 wt-%, based on the weight of the ready to use composition.

Long Chain Fatty Acid

Illustrative examples of long chain fatty acids that can be used herein include C₁₀ to C₂₂ mono-and poly-unsaturated fatty acids and C₈ to C₁₆ saturated fatty acids. Exemplary unsaturated fatty acids include, but are not limited to, undecylenic acid, myristoleic acid, palmitoleic acid, oleic acid, linoleic acid and linolenic acid. Exemplary saturated fatty acids include, but are not limited to, caprylic acid, capric acid, lauric acid, and myristic acid, and palmitic acid. Other examples of suitable long chain fatty acids include mixtures derived from oils such as sunflower fatty acid and soya fatty acid, e.g., NOURACID™ HE 30 and NOURACID™ SE 30, both from Akzo Nobel.

One or more long chain fatty acids may be used in the compositions of the present invention at a suitable level to produce the desired result. In a preferred embodiment long chain fatty acids are present in a total amount of at least 0.1 wt-%, more preferably at least 0.25 wt-%, and even more preferably at least 0.5 wt-%, and most preferably at least 1.0%, based on the ready to use composition. In a preferred embodiment, the acids are present in a total amount of no greater than 10 wt-%, more preferably no greater than 5 wt-%, and even more preferably no greater than 2 wt-%, based on the total weight of the ready to use composition.

Optional Additives

In some embodiments, the article of the invention will further comprise optional additives for the purpose of providing some desired performance. For example, chew articles of the invention may further comprise additives for imparting desired scent, odor, and flavors, color, etc.

Articles of the invention may contain additional compatible pharmaceutically active materials for combination therapy (such as supplementary antimicrobials, anti-caries agents, anti-parasitic agents, vitamins, antipruritics, astringents, local anesthetics, steroids, non-steroidal anti-inflammatory agents, or other anti-inflammatory agents), or may contain materials useful in physically formulating various dosage forms of the present invention, such as excipients, dyes, perfumes, lubricants, mold release agents, fibers, plasticizers, stabilizers, flavoring agents, preservatives, or antioxidants.

Articles of the invention may further include fibers such as cellulose fiber, wheat bran, innulin, wood fiber, sisal fiber, and mixtures thereof. The fibers may be added in amounts up to 60% by weight of the composition. The fibers are added as filler and to adjust the texture of the chew article where the ease of chewing the gluten matrix is generally increased by the addition of fiber. The fiber content is preferably from 1% to 20% of the composition by total weight. Starches that resist digestion can also be added as filler materials. Highly cross-linked wheat starch manufactured, for example, by processes disclosed in U.S. Pat. Nos. 5,855,946 and 6,299,907 (both Seib et al.) is representative. Such fillers reduce the overall caloric content of the chew article, making them less fattening for the animal.

It will also be appreciated that additional antiseptics, disinfectants, or antibiotics may be included and are contemplated. These include, for example, addition of metals or metal ions such as silver, copper, zinc; chlorhexidine and its various salts such as chlorhexidine digluconate; polyhexamethylenebiguanide, parachlorometaxylenol, triclosan, and antimicrobial quaternary amines including polymeric quaternary amines.

Enzymes

In some embodiments, chews of the invention will further comprise therapeutic enzymes.

Enzymes useful in the practice of this invention are therapeutic enzymes, which herein means that they cause (e.g., by catalysis) the decomposition of harmful carbohydrates, proteins, lipids, and/or bacterial substrates in the mouth of a subject (e.g., in the oral plaque and saliva). A preferred group of enzymes generate bactericidal products (e.g., H₂O₂). A second preferred group of enzymes are capable of hydrolyzing and solubilizing the carbohydrate biofilm that plaque-causing bacteria generate.

Optionally, one or more enzyme substrates can be incorporated in articles of the present invention to enhance the therapeutic function of the enzymes. For example, for systems requiring water for activation, an enzyme substrate could be present in the composition with the enzymes if the system is anhydrous until use, thereby keeping the enzymes and substrate from interacting.

It is advantageous to use enzymes that are substantially active at a pH prevailing in the mouth. Typically, this is about pH 5.0 to about pH 9.0, more typically about pH 6.0 to about pH 8.5, and even more typically about pH 6.4 to about pH 7.5. Under certain conditions the pH may be significantly lower, which allows for the use of a wider variety of enzymes.

Oxidoreductase and hydrolase enzymes are useful classes of enzymes for use in the present invention. Depending on the desired effect, any of the various types of enzymes can be preferred for a particular embodiment.

Oxidoreductase enzymes are those classified under the Enzyme Classification number E.C. 1 in accordance with the Recommendations (1992) of the International Union of Biochemistry and Molecular Biology (IUBMB). They catalyze oxidoreductions (i.e., redox reactions). Within the group of oxidoreductase enzymes are oxidase enzymes, peroxidase enzymes, and laccase enzymes.

Oxidase enzymes catalyze the oxidation of a substrate by acting on O₂ as an acceptor of electrons and forming hydrogen peroxide. Such enzymes are classified under the enzyme classification E.C. 1.1.3, E.C. 1.2.3, E.C. 1.3.3, E.C. 1.4.3, E.C. 1.5.3. E.C. 1.7.3, E.C. 1.8.3, E.C. 1.9.3. Examples include, but are not limited to, glucose oxidase, sucrose oxidase, lactate oxidase, (S)-2-hydroxy-acid oxidase, hexose oxidase, L- or D-amino-acid oxidase, xylitol oxidase, xanthine oxidase, glycolate oxidase, L-sorbose oxidase, alcohol oxidase, gulonolactone oxidase. Corresponding enzyme substrates include, but are not limited to, beta-D-glucose, sucrose, lactate, (S)-2-hydroxy-acid, broad spectrum of carbohydrates including D-glucose, D-galactose, D-mannose, maltose, lactose, and cellobiose, etc., L-or D-amino acids, xylitol, xanthine, alpha-hydroxy acids, L-sorbose, a primary alcohol, and L-gulono-1,4-lactone.

Peroxidase enzymes act on peroxide as an acceptor of electrons. These include enzymes classified under the enzyme classification E.C. 1.1 1. The different types of peroxidase enzymes are distinguished by the donor molecules from which they take electrons to donate to hydrogen peroxide. In accordance with the present invention a peroxidase is used to generate free radicals from donor molecules. The donor molecules are typically capable of acting as a substrate for the peroxidase in generating such free radicals. Examples include, but are not limited to, horseradish peroxidase, soybean peroxidase, polyphenol peroxidase, manganese peroxidase, L-ascorbate peroxidase, chloroperoxidase, and iodide peroxidase. Corresponding enzyme substrates include, but are not limited to, hydrogen peroxide and electron donor molecules such as polyphenol, manganese (II), ascorbic acid, chloride, and iodide.

Laccase enzymes act on O₂ and yield water without any need for peroxide. These include enzymes classified under the enzyme classification E.C. 1.10.3. Corresponding substrates include, but are not limited to, O-and P-quinols, aminophenols, and phenylenediamine.

Hydrolase enzymes are those classified under the Enzyme Classification number E.C. 3 in accordance with the Recommendations (1992) of the International Union of Biochemistry and Molecular Biology (IUBMB). Within the group of hydrolase enzymes are protease enzymes, carbohydrase enzymes, and lipase enzymes. Preferred hydrolase enzymes are carbohydrases.

Protease enzymes act to break down or hydrolyze proteins. Such enzymes are classified under the classification E.C. 3.4.21, E.C. 3.4.22, E.C. 3.4.23, E.C. 3.4.24. Example include, but are not limited to, trypsin, papain, pancreatin, pepsin (e.g., pepsin A, pepsin B), chymosin, cathepsin E, gastricsin, cathepsin D, phytepsin, cyprosin, cardosin A, cardosin B, nephentesin, neurosporapepsin, saccharopepsin, renin, plasmepsin, rhodorulapepsin, acrocyclindropepsin, pycnoporopepsin, physaropepsin, aspergillopepsin, penicillopepsin, rizopuspepsin, mucorpepsin, polyproppepsin, candidaparapsin, candidapepsin, yapsin 1, yapsin 2, yapsin 3, pseudomonaspepsin, xanzhornonpepsin, thermopsin, scytalidopepsin, aleurain, omptin, lysosomale, carboxypeptidase A, cathepsin A, lysosomale pro-X carboxypeptidase, asparaginyl endopeptidase, y-glutamylhydrolase, bacillus pepstatin insensitive acid endopeptidase, carboxypeptidase, and insulysin. Corresponding substrates for proteases are various proteins and peptides. Proteases can be used in combination with zinc to enhance anti-plaque functions. Proteases break down salivary proteins, which are adsorbed onto the tooth surface and form the pellicle, the first layer of resulting plaque. Proteases along with lipases destroy bacteria by lysing proteins and lipids which form the structural components of bacterial cell walls and membranes.

Carbohydrase enzymes act to break down or hydrolyze carbohydrates. Such enzymes are classified under the classification E.C. 3.2.1. Examples include, but are not limited to, dextranase, mutanase, cellulase, amylase, alpha-glucosidase, beta-glucosidase, lactase, invertase, amyloglucosidase, and lysozyme. Corresponding substrates for the carbohydrase enzymes include, but not limited to, dextran, cellulose, starch, oligosaccharides, beta-D-glucosides, lactose, sucrose, polysaccharides, and bacterial cell wall. Of these carbohydrases, dextranase, mutanase, and amylase, alone or in combination are preferred for use. Dextranase and mutanase break down the carbohydrate biofilm structure produced by bacteria that forms a matrix for bacterial adhesion. Thus they not only solubilize plaque, but they also allow the antimicrobial lipid component access to the bacteria within this matrix. Amylases in combination with proteases, not only prevent plague formation, but also prevent the development of calculus by breaking-up the carbohydrate-protein complex that binds calcium, preventing mineralization.

Lipase enzymes act to break down or hydrolyze fatty substances, e.g., fatty acids and fatty acid esters. Such enzymes are classified under classification E.C. 3.1.1 and E.C. 3.1.4. Examples include, but are not limited to, Lipase 4000, Lipase B, Lipase 448, gastric lipase, pancreatic lipase, and plant lipase. Corresponding substrates for lipase enzymes are various fats and oils.

Various combinations of enzymes and optional substrates can be used to enhance therapeutic functions. Examples include: combinations of various oxidoreductase enzymes and their corresponding substrates; combinations of glucose oxidase, glucose, and thiocyanate; and combinations of glucose oxidase/glucose dehydrogenase and glucose.

Chews of the invention can be prepared with desired amounts of enzyme to achieve the desired therapeutic effect. Chews may include one or more enzymes and optionally one or more enzyme substrates taking into account both the activity of the enzyme preparation as well as its total amount. Generally, formulation will be based on activity, not on total weight of enzyme preparation. The level of enzyme used in the practice of this invention will depend on the enzymatic activity of the enzyme and the desired therapeutic effect.

Enzymes can be used in soluble form or immobilized form. An immobilized enzyme may be used to enhance enzymatic stability and reactivity. There are many methods available for immobilization including binding on prefabricated carrier materials and incorporating into in situ prepared carriers. Operative binding forces vary between weak multiple adsorptive interactions and single attachments through strong covalent binding. The appropriate methods depend on the enzyme structure and application. In general, enzymes can be immobilized by attachment to carriers through either chemical reaction or physical absorption and can be used in a variety of methods as described in W. Tischer, F. Wedekind, Topics in Current Chemistry, Vol. 200, Springer, Berlin Heidelberg, 1999. Alternatively, enzymes can be encapsulated within a membrane or liposome/micelle.

The above summary of the present invention is not intended to describe each disclosed embodiment or every implementation of the present invention. In several places throughout the application, guidance is provided through lists of examples, which examples can be used in various combinations. In each instance, the recited list serves only as a representative group and should not be interpreted as an exclusive list.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A pet chew article comprising: (a) edible chew base, (b) an effective amount of antimicrobial lipid, and (c) at least one of (1) an effective amount of tartar control agent or (2) an effective amount of a long chain fatty acid.

2. The article of claim 1 wherein said edible chew base is selected from the group consisting of wheat gluten, gelatinized starch, and combinations thereof.

3. The article of claim 1 wherein said antimicrobial lipid is selected from the group consisting of fatty acid ester of a polyhydric alcohol, a fatty ether of a polyhydric alcohol, alkoxylated derivatives thereof (of either the ester or ether), and combinations thereof.

4. The article of claim 1 wherein said antimicrobial lipid is selected from the group consisting of glycerol monolaurate, glycerol monocaprate, glycerol monocaprylate, propylene glycol monolaurate, propylene glycol monocaprate, propylene glycol monocaprylate, and combinations thereof.

5. The article of claim 1 wherein said tartar control agent is selected from the group consisting of an alpha-hydroxy acid, a beta-hydroxy acid, a chelating agent, and combinations thereof.

6. The article of claim 5 wherein said alpha-hydroxyl acid is represented by the formula:
R⁵(CR⁶OH)ₙCOOH
wherein: R⁵ and R⁶ are each independently H or a C₁ to C₈ alkyl group (straight, branched, or cyclic), a C₆ to C₁₂ aryl, or a C₆ to C₁₂ aralkyl or alkaryl group (wherein the alkyl group is straight, branched, or cyclic), R⁵ and R⁶ may be optionally substituted with one or more carboxylic acid groups; and n is 1 to 3.

7. The article of claim 5 wherein said beta-hydroxyl acid is represented by the formula: 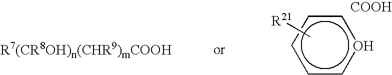
wherein: R⁷, R⁸, and R⁹ are each independently H or a C₁ to C₈ alkyl group (saturated straight, branched, or cyclic group), a C₆ to C₁₂ aryl, or a C₆ to C₁₂ aralkyl or alkaryl group (wherein the alkyl group is straight, branched, or cyclic), wherein R⁷ and R⁸ may be optionally substituted with one or more carboxylic acid groups; m is 0 or 1; n is 1 to 3,; and R²¹ is H, a C1 to C4 alkyl, or a halogen.

8. The article of claim 5 wherein said chelating agent is selected from the group consisting of ethylene diamine tetraacetic acid and salts thereof, adipic acid, succinic acid, polyphosphoric acid, sodium acid pyrophosphate, sodium hexametaphosphate, acidified sodium hexametaphosphate, nitrilotris(methylenephosphonic acid), diethylenetriaminepentaacetic acid, 1-hydroxyethylene-1,1-diphosphonic acid, diethylenetriaminepenta-(methylenephosphonic acid), and combinations thereof.

9. The article of claim 1 wherein said long chain fatty acid is selected from the group consisting of C₁₀ to C₂₂ mono-and poly-unsaturated fatty acids, C₈ to C₁₆ saturated fatty acids, and combinations thereof.

10. The article of claim 1 wherein said long chain fatty acid is selected from the group consisting of undecylenic acid, myristoleic acid, palmitoleic acid, oleic acid, linoleic acid, linolenic acid, caprylic acid, capric acid, lauric acid, myristic acid, palmitic acid, and combinations thereof.

11. The article of claim 1 further comprising a therapeutic enzyme.

12. The article of claim 11 wherein said enzyme is selected from the group consisting of: oxidoreductase enzymes, hydrolase enzymes, or mixtures thereof.

13. The article of claim 11 further comprising an enzyme substrate.

14. A process for the prevention of tartar accumulation on the teeth of a dog or cat, comprising chewing or eating by the dog or cat of an article of claim 1.
